(19) United States
(12) Patent Application Publication
(10) Pub. No.: US 2017/0213014 A1
(43) Pub. Date: Jul. 27, 2017

(54) SYSTEM AND METHOD FOR DETECTING SMOKING BEHAVIOR

(71) Applicant: Covidien LP, Mansfield, MA (US)

(72) Inventor: Mark Yu-Tsu Su, Boulder, CO (US)

(21) Appl. No.: 15/004,532

(22) Filed: Jan. 22, 2016

(51) Int. Cl. G06F 19/00; A61B 5/00; A61B 5/11

(52) U.S. Cl. CPC G06F 19/36; A61B 5/1123; A61B 5/6824; A61B 2562/0219

(57) ABSTRACT

Methods, apparatuses and systems for detecting smoking behavior of a user are described. Methods may include receiving accelerometry data from at least one accelerometer coupled with the user and detecting a pattern of movement from the accelerometry data indicative of smoking behavior. Described methods may also include detecting a plurality of sub-patterns of movement such as repeatedly swinging at least one hand toward and away from the user's face in a sequence indicative of smoking behavior. An apparatus for detecting smoking behavior of a user may include a processor, memory in electronic communication with the processor, and instructions stored in the memory and operable, when executed by the processor, to cause the apparatus to receive accelerometry data from at least one accelerometer and detect a pattern of movement from the accelerometry data indicative of smoking behavior.

### BACKGROUND

Smoking is the primary risk driver for a variety of respiratory diseases including lung cancer and chronic obstructive pulmonary disease (COPD). The most effective component of lung cancer and COPD treatment therapies is to quit smoking, and patients may enroll in a smoking cessation plan to help them quit. Typical smoking cessation plans include a social support or counseling component and may also include prescription medicine such as nicotine replacement therapy (NRT) or non-nicotine drugs that reduce withdrawal symptoms or block the effects of nicotine.

Accurate measures of smoking behavior such as smoking frequency may be helpful in monitoring the patient's progress or to modify the dosage of NRT medication. However, current smoking cessation plans typically rely on subjective reporting of smoking behavior by the patient, which may be unreliable due to inaccurate reporting or non-compliance. Accordingly, there may be a need for objective measurements of smoking behavior that are accurate and require little or no input by the patient.

### SUMMARY

The described features generally related to methods, devices, and systems for detecting smoking behavior of a user from accelerometry data. The accelerometry data may be collected passively from a device worn or otherwise coupled with the user, and the data may be processed locally on the device or may be transmitted to a remote device for processing. Processing the accelerometry data generally includes detecting patterns of movement that are indicative of smoking behavior. Once smoking behavior has been detected, a notification may be transmitted to the user or a clinician responsible for the user.

In certain embodiments described herein, a method for detecting smoking behavior of a user includes receiving accelerometry data from an accelerometer coupled with the user and detecting a pattern of movement from the accelerometry data indicative of smoking behavior. The pattern of movement may include a single pattern (e.g., repeatedly swinging a hand toward and away from the user's face) or may include multiple sub-patterns detected in a sequence that is indicative of smoking (e.g., a flicking motion of one hand after repeatedly swinging the hand toward and away from the user's face).

In some described embodiments, detecting a pattern of movement indicative of smoking behavior may include dividing the received accelerometry data into temporal segments (e.g., 5 minutes), analyzing the acceleration peaks within each temporal segment, and assigning a probability of smoking behavior for each temporal segment based on a comparison between the received accelerometry data and predetermined accelerometry data that is known to be indicative of smoking behavior.

Smoking behavior may be detected with other sensors in addition to an accelerometer. For example, smoke, heat, light, or sound may be detected with one or more sensors, which may increase the accuracy of smoking behavior detection over using accelerometry data alone.

Apparatuses and systems for detecting smoking behavior of a user are also described. In some embodiments, an apparatus includes a processor and instructions stored in memory configured to cause the processor to receive accelerometry data from at least one accelerometer and detect a pattern of movement from the accelerometry data indicative of smoking behavior. In other embodiments, a system for detecting smoking behavior of a user includes a wearable apparatus with at least one accelerometer, a computing apparatus configured to detect a pattern of movement indicative of smoking behavior from accelerometry data received from the wearable apparatus, and a display configured to display a notification that smoking behavior of the user has been detected.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the embodiments may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an example of a wireless sensor system that supports smoking behavior detection in accordance with aspects of the present disclosure;

FIG. 2 illustrates a schematic of patterns of motion indicative of smoking behavior in accordance with aspects of the present disclosure;

FIG. 3 illustrates a graphical representation of accelerometry data for smoking behavior detection in accordance with aspects of the present disclosure;

FIG. 4 illustrates an example of a device that supports smoking behavior detection in accordance with aspects of the present disclosure;

FIG. 5 illustrates an example of a device that supports smoking behavior detection in accordance with aspects of the present disclosure;

FIG. 6 illustrates an example of a device that supports smoking behavior detection in accordance with aspects of the present disclosure;

FIG. 7 illustrates a method for smoking behavior detection in accordance with aspects of the present disclosure;

FIG. 8 illustrates a method for smoking behavior detection in accordance with aspects of the present disclosure; and

FIG. 9 illustrates a method for smoking behavior detection in accordance with aspects of the present disclosure.

### DETAILED DESCRIPTION

The described methods, systems, and apparatuses generally relate to detecting smoking behavior of a user. The smoking behavior such as smoking frequency, duration, time of day, and proximity to other activities or people (i.e., triggers) may all be accurately detected and then reported to a clinician who may use the data to monitor the user's progress and assist the user in smoking cessation. As described herein, the smoking behavior of a user may be detected by analyzing accelerometry data received from one or more devices worn by the user. Detecting smoking behavior from one or more patient-worn devices may ensure more accurate and reliable data than subjective reporting because the data is collected continuously and passively with little or no input required by the user.

The accelerometry data can be analyzed to determine certain patterns of movement that are, either alone or in combination with other patterns of movement, indicative of smoking behavior. For example, repeatedly swinging one hand towards and away from the user's face may indicate that the user is actively smoking. Other patterns of movement may also be detected to improve the specificity of the detection methods. For example, the probability of smoking behavior may be greater if the swinging motion was detected after a motion indicative of removing a cigarette from a package and lighting it up was detected or before a staccato flicking motion indicative of discarding the ashes of the cigarette was detected.

In some examples, the specificity of smoking behavior detection may be increased by incorporating sensor data other than accelerometry data into the detection method. For instance, one or more sensors may detect smoke, heat, light, or sound, which may indicate smoking behavior either alone or in conjunction with certain patterns of motion.

The smoking behavior data may be displayed to the user as part of an application that generally tracks activities and health information of the user. The application may include a dashboard that illustrates smoking behavior statistics (e.g., frequency, duration, time of day) and may also include a social media aspect that notifies others within a support or peer group when smoking behavior has been detected. The application may also allow for manual inputs by the user related to a smoking cessation program or other health-related information generally. For example, a user may input information related to NRT medication (e.g., the dosage and the time of day taken), which may be correlated with the smoking behavior detected from the accelerometry data.

The smoking behavior data may also be displayed to a clinician as part of a smoking cessation program that the user enrolls in. For example, the smoking behavior data may be sent to a clinician for monitoring purposes or to a pharmacists to adjust the dosage of smoking cessation medication (e.g., the level of nicotine in NRT medication).

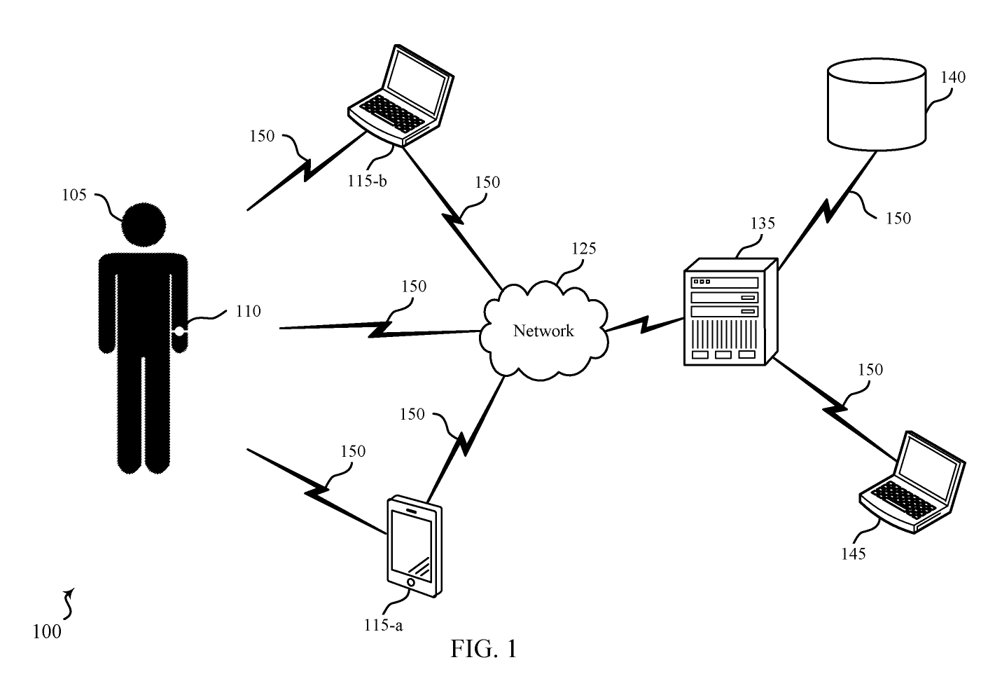

With reference to FIG. 1, an example of a wireless monitoring system 100 is illustrated in accordance with various aspects of the present disclosure. The system 100 includes a user 105 wearing, carrying, or otherwise physically coupled with a sensor unit 110. In accordance with various embodiments described herein, the sensor unit 110 may collect accelerometry data from movements by the user 105 and transmit the data via wireless communications links 150 to local computing devices 115-a, 115-b or to a server 135 via a network 125 such as the Internet. The data collected by the sensor unit 110 may also be conveyed from the server 135 to a remote computing device 145 or a remote database 140. Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

The sensor unit 110 may include one or more sensors configured to collect information related to the location and movement of the user 105 as well as a variety of physiological parameters. For example, the sensor unit 110 may include one or more accelerometers configured to collect 3-axis Cartesian accelerometry data. In certain aspects, the sensor unit 110 may include additional sensors such as a pulse oximetry (SpO2) sensor, a heart rate sensor, a blood pressure sensor, an electrocardiogram (ECG) sensor, a respiratory rate sensor, a glucose level sensor, a body temperature sensor, a global positioning sensor (GPS), or any other sensor configured to collect physiological, location, or motion data. In addition, the sensor unit 110 may include one or more sensors configured to detect smoke, heat (i.e., thermal energy), light, or sound such as a smoke detector, a heat sensor (e.g., infrared sensor), an optical sensor, or an audio sensor.

Although a single sensor unit 110 is shown, multiple sensor units 110 may be worn by the user 105 and may be in electronic communication with each other (e.g., one on each wrist). The sensor unit 110 may be physically coupled with the user 105 in a variety of ways depending on the data being collected. For example, the sensor unit 110 may be worn around the user's wrist, attached to the user's finger, or coupled to the user's chest.

Local computing device 115-a may be a wireless device such as a tablet, cellular phone, personal digital assistant (PDA), dedicated receiver, or other similar device. Local computing device 115-b may be a wireless laptop computer or mobile computer station also configured to receive signals from the sensor unit 110. In accordance with various embodiments, the local computing devices 115 may be configured to wirelessly receive data from the sensor unit 110 such as accelerometry data. As described below, the accelerometry data may be processed at any of the sensor unit 110, the local computing devices 115, the server 135, or the remote computing device 145 to detect patterns of motion indicative of smoking behavior. In any case, once smoking behavior is detected, a notification may be transmitted and displayed on the remote computing device 145, which may be used by a clinician to remotely monitor the smoking behavior of the user 105. In addition, a notification of smoking behavior may be displayed on the local computing devices 115 for review by the user 105.

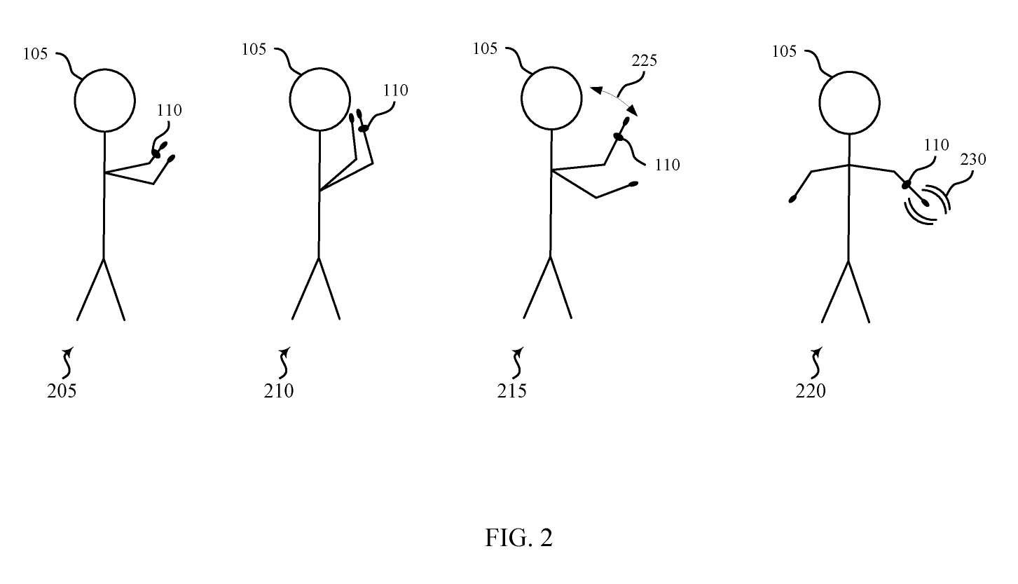

With reference to FIG. 2, a schematic of a user 105 performing motions indicative of smoking behavior is illustrated in accordance with various aspects of the present disclosure. A typical smoking event may include one or more patterns of movement that are indicative of smoking behavior either alone or in combination with one or more other patterns of movement. For example, in motion 205, the user 105 is bringing both hands together at a location away from the user's face. This motion may be detected with one or more sensor units 110 as described in more detail below. The motion 205 of bringing both hands together away from the user's face may occur during the process of opening a package of cigarettes or removing a cigarette (or e-cigarette) from its packaging.

In motion 210, the user 105 is bringing both hands together near the user's face. This motion may be detected with one or more sensor units 110 and may occur for example while the user 105 is lighting a cigarette for the first time, which requires both hands to be near the user's face (i.e., one hand to hold the cigarette while the other hand lights the cigarette). In addition to the proximity of both hands to the user's face, the process of lighting a cigarette may also be detected by detecting one or more staccato vibrations resulting from the thumb engaging the lighter.

It may be appreciated that the terms “near” and “away” as used herein to describe the proximity of the user's hands to the face may be readily understood in the context of the motions being described and are not readily defined by precise numerical ranges. For example, in motion 210, bringing both hands together near the user's face may refer to a distance from the user's face that would allow one hand to hold the cigarette in the user's mouth while the other hand lights the cigarette. Similarly, bringing both hands together at a location away from the user's face may refer to a distance that is at least greater than the “near” proximity just described.

In motion 215, the user 105 is repeatedly moving at least one hand toward and away from the user's face as indicated by arrow 225. This motion 215 may be detected by one or more sensor units 110 as a repeated swinging motion. The motion 215 may occur for example while the user 105 is repeatedly bringing a cigarette to the user's face to inhale and then moving the cigarette away from the face to exhale.

In motion 220, the user 105 is flicking or twitching at least a portion of one hand as indicated by lines 230, and my occur as the user 105 is flicking the ashes from the cigarette. This motion 220 may be detected by one or more sensor units 110 as a staccato, vibration, or otherwise pulsating motion.

In accordance with various embodiments, the smoking behavior of a user 105 may be detected by detecting any of these motions 205, 210, 215, or 220 individually or in combination. In some examples, smoking behavior may be detected by detecting one or more sub-patterns of movement in a sequence indicative of smoking behavior. For example, in some embodiments, smoking behavior may only be detected after detecting a repeated swinging motion 215 followed by or intermittent with a flicking motion 220. Detecting multiple sub-patterns of motion in a particular sequence may increase the specificity of smoking behavior detection over detecting just a single pattern of motion. For example, detecting a swinging motion or that both hands are together near the user's face may occur while the user 105 is eating, drinking, or applying makeup. Thus, certain patterns of motion (e.g., the staccato motion 220) that are relatively unique to the act of smoking may be used as filters to avoid mistaking other activities for actual smoking behavior.

In some aspects, smoking behavior of a user 105 may be detected after detecting both hands of the user coming together away from the user's face followed by a repeated swinging motion indicating at least one of the user's hands moving toward and away from the user's face. In another example, smoking behavior of a user 105 may be detected after detecting both hands of the user 105 coming together near the user's face followed by a repeated swinging motion indicating at least one of the user's hands moving toward and away from the user's face.

In certain examples, the smoking behavior of a user 105 may be detected with one or more sensors in addition to an accelerometer. For example, an inhalation of a user 105 may be detected with an SpO2 sensor after detecting both hands of the user coming together near the user's face. Detecting a deep or sustained inhalation may increase the specificity of the smoking behavior detection method by filtering out other activities that involve brining both hands together near the face such as eating or drinking. In other examples, light, heat, or smoke may be detected after detecting both hands coming together near the user's face, which may also increase the likelihood that the user 105 is actually lighting a cigarette as opposed to eating or drinking. Additionally or alternatively, an audio sensor may detect certain sounds indicative of smoking behavior such as the clicking or flicking noise of a lighter.

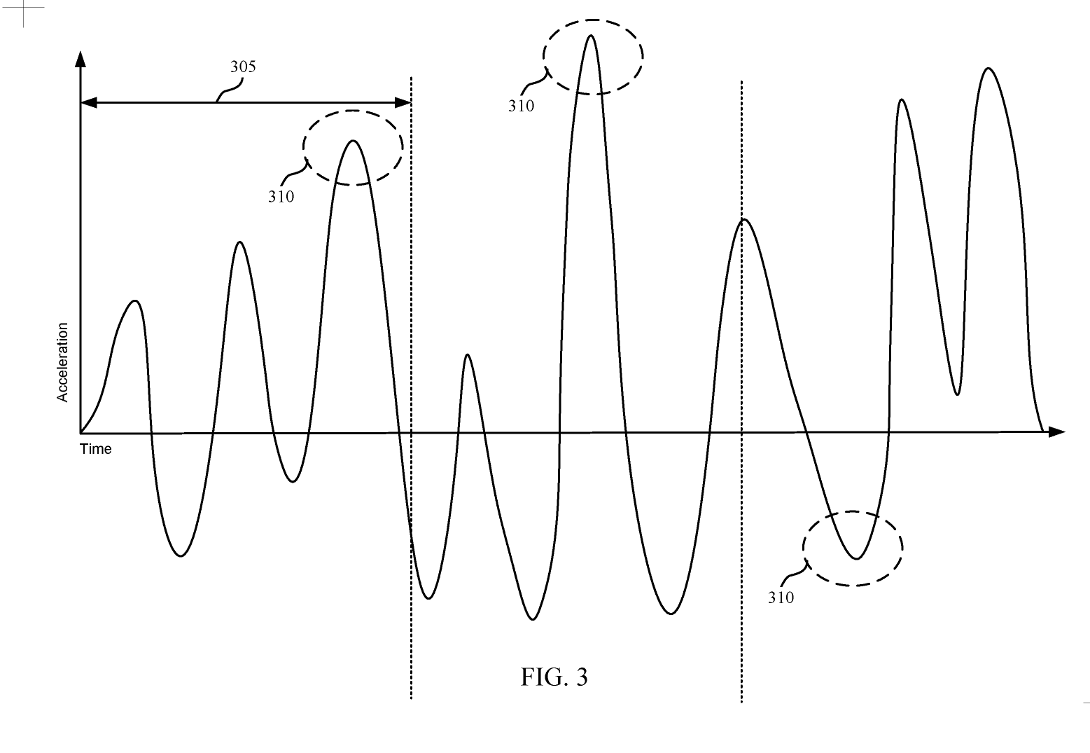

With reference to FIG. 3, an exemplary accelerometry data set is illustrated in accordance with various embodiments. The illustrated accelerometry data may be collected by a sensor unit 110 and processed locally by the sensor unit 110 or any of a local computing device 115, a server 135, or a remote computing device 145, as described with reference to FIG. 1. The accelerometry data set may be represented graphically with acceleration values plotted on the vertical axis and time plotted along the horizontal axis. It may be appreciated that by processing the accelerometry data, the motion and location of one or both of the user's hands may be determined at various points in time, thereby facilitating the recognition of patterns of movement indicative of smoking behavior, as described with reference to FIG. 2. In some embodiments, the accelerometry data is collected in 3-axis Cartesian coordinates (e.g., x, y, z) and then converted into spherical coordinates (e.g., r, φ, θ).

The accelerometry data may be divided into time segments 305 with a predetermined duration (e.g., 5 minutes) within which a smoking event may have occurred. Within each time segment 305, one or more accelerometry peaks 310 may be identified. As may be appreciated, the peaks 310 may indicate certain movements such as changes in directions (e.g., swinging motion), sudden stops in motion (e.g., bringing the hands together), or pulsating motions (e.g., staccato motion of lighting or flicking the ashes from a cigarette).

Processing the accelerometry data may also include determining certain information regarding the acceleration values at each peak 310. For example, the information determined for each peak 310 may include the temporal location of each peak 310 (e.g., the occurrence time), the duration of acceleration, the direction of acceleration (e.g., the theta angle direction of acceleration and the phi angle direction of acceleration), and the amplitude of acceleration. The determined acceleration information may be compiled into a database for further analysis.

Processing the accelerometry data may further include comparing the determined acceleration information for each peak 310 to predetermined acceleration information that is known to be indicative of smoking behavior. Finally, for each temporal segment 305, a probability of smoking behavior may be assigned based on comparing the received accelerometry data to the predetermined accelerometry data. As may be appreciated, the probability of smoking behavior may be based on the degree of similarity between the received accelerometry data and the predetermined accelerometry data. In addition, the probability may be adjusted after incorporating additional sensor information such as the presence of light, heat, smoke, or sounds that are indicative of smoking behavior.

The predetermined acceleration information may be derived from one or more machine learning algorithms that associates certain accelerometry patterns to smoking behavior. For example, to train the probability determining algorithm, raw accelerometry data from a user 105 smoking a cigarette may be input, converted, and divided into temporal segments 305 as described above. Furthermore, the acceleration peaks 310 within each temporal segment 305 may be identified, and acceleration information for each peak 310 may be determined as described above. If smoking behavior is actually occurring during one or more temporal segments 305, the acceleration information within those temporal segments 305 may be flagged as indicative of smoking behavior. It may be appreciated that the specificity of the algorithm may be increased by comparing the accelerometry data of a user 105 smoking a cigarette against other activities such as eating, drinking, or applying makeup. The machine learning algorithm may be tailored for a specific user 105 and may adapt over time or may be based on a population of users 105.

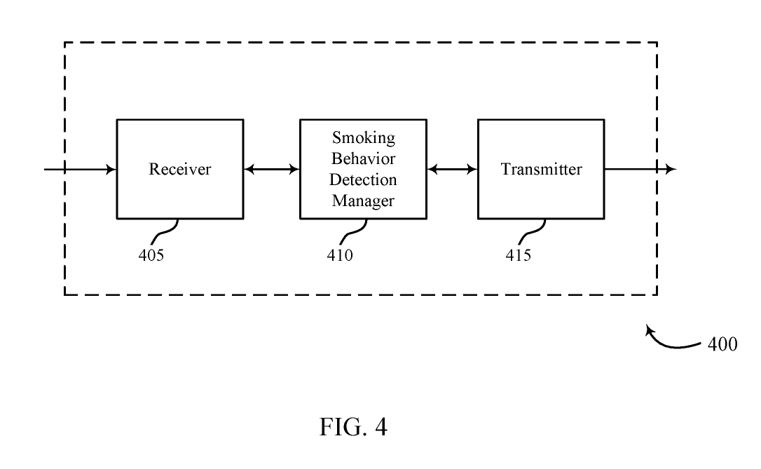

FIG. 4 illustrates a block diagram of a device 400 that supports smoking behavior detection in accordance with various aspects of the present disclosure. The device 400 may communicate via wired or wireless means (e.g., wireless links 150) with a sensor unit 110 and may be an example of aspects of a local computing device 115, a server 135, or a remote computing device 145, as described with reference to FIG. 1. Alternatively, the device 400 may be incorporated into a sensor unit 110 for local processing of accelerometry data. The device 400 may include a receiver 405, a smoking behavior detection manager 410, and a transmitter 415. In accordance with various embodiments, the device 400 may be operable to detect smoking behavior of a user 105 by detecting one or more patterns of movement that are indicative of smoking behavior from received accelerometry data.

The receiver 405 may receive information such as packets, user data, or control information associated with various sensor units 110 (e.g., an accelerometer). For example, the receiver 405 may receive accelerometry data from a sensor unit 110 having an accelerometer coupled with a user 105 as described with reference to FIGS. 1 and 2. The receiver 405 may receive data via wireless or wired means, and may pass the received data on to other components within device 400 (e.g., to the smoking behavior detection manager 410).

The smoking behavior detection manager 410 may include circuitry, logic, hardware and/or software for detecting a pattern of movement from the received accelerometry data that is indicative of smoking behavior. For example, the smoking behavior detection manager 410 may detect certain patterns of movement (e.g., repeatedly swinging at least one hand toward and away from the user's face) that are, either alone or in combination with other patterns of movement, indicative of smoking behavior, as described with reference to FIG. 2. Additionally or alternatively, the smoking behavior detection manager 410 may process the received accelerometry data into temporal segments 305, identify a plurality of acceleration peaks 310 within each temporal segment 305, and assign a probability of smoking behavior to each temporal segment 305 based on a comparison algorithm as described with reference to FIG. 3. In certain embodiments, the smoking behavior detection manager 410 incorporates additional information other than accelerometry data (e.g., smoke detector data) into the smoking behavior detection method.

The transmitter 415 may transmit signals received from other components of device 400 via wired or wireless means to one or more other devices (e.g., to a server 135), as described with reference to FIG. 1. In some examples, the transmitter 415 may transmit processed accelerometry data or an indication (e.g., a notification) of smoking behavior from the smoking behavior detection manager 410. Additionally or alternatively, the transmitter 415 may transmit the raw accelerometry data received from receiver 405 for additional processing on one or more other devices (e.g., a server 135).

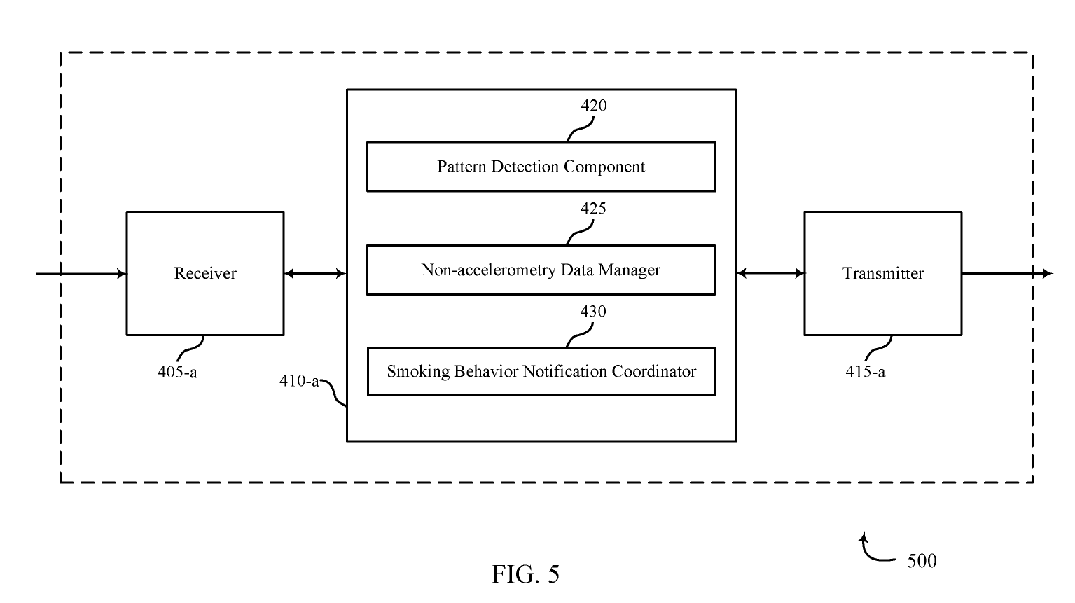

FIG. 5 illustrates a block diagram of a device 500 that supports smoking behavior detection in accordance with various aspects of the present disclosure. The device 500 may be an example of aspects of device 400 as described with reference to FIG. 4 or aspects of a sensor unit 110, a local computing device 115, a server 135, or a remote computing device 145 as described with reference to FIG. 1. The device 500 may include a receiver 405-a, a smoking behavior detection manager 410-a, and a transmitter 415-a, which may each be examples of aspects of the receiver 405, smoking behavior detection manager 410, and transmitter 415 described with reference to FIG. 4. Furthermore, the smoking behavior detection manager 410-a may include a pattern detection component 420, a non-accelerometry data manager 425, and a smoking behavior notification coordinator 430. In accordance with various embodiments, the device 500 may be operable to detect smoking behavior of a user 105 by detecting one or more patterns of movement that are indicative of smoking behavior from received accelerometry data.

The pattern detection component 420 may include circuitry, logic, hardware and/or software for detecting a pattern of movement from the received accelerometry data that is indicative of smoking behavior. For example, the pattern detection component 420 may detect certain patterns of movement (e.g., repeatedly swinging at least one hand toward and away from the user's face) that are, either alone or in combination with other patterns of movement, indicative of smoking behavior, as described with reference to FIG. 2.

In some embodiments, the pattern detection component 420 detects a plurality of sub-patterns of movement in a sequence that is indicative of smoking behavior. In an example, the pattern detection component 420 may detect a vibration motion of one hand of the user 105 that is indicative of the user 105 flicking the hand (e.g., motion 220) after detecting a repeating swinging motion of the hand that is indicative of the hand moving toward and away from the user's face (e.g., motion 215).

In another example, the pattern detection component 420 may detect a repeating swinging motion of one hand of the user 105 that is indicative of the hand moving toward and away from the user's face (e.g., motion 215) after detecting a motion of both hands of the user 105 that is indicative of both hands coming together away from the user's face (e.g., motion 205).

In yet another example, the pattern detection component 420 may detect a repeating swinging motion of one hand of the user 105 that is indicative of the hand moving toward and away from the user's face (e.g., motion 215) after detecting a motion of both hands of the user 105 that is indicative of both hands coming together near the user's face (e.g., motion 210). It may be appreciated that the patterns of movement described herein and there particular sequences are exemplary and that additional patterns of movement other than those described may be used to detect the smoking behavior of a user 105.

Additionally or alternatively, the pattern detection component 420 may process the received accelerometry data into temporal segments 305 and assign a probability of smoking behavior to each temporal segment 305 based on a comparison algorithm as described with reference to FIG. 3. For example, the pattern detection component 420 may convert 3-axis Cartesian accelerometry data received from receiver 405-a into spherical coordinate accelerometry data and divide the spherical coordinate accelerometry data into a plurality of temporal segments 305 based on a predetermined duration (e.g., 5 minutes). The pattern detection component 420 may also identify a plurality of acceleration peaks 310 within each temporal segment 305, as described with reference to FIG. 3.

After identifying a plurality of acceleration peaks 310, the pattern detection component 420 may then determine, for each of the plurality of acceleration peaks, acceleration information such as an occurrence time, a duration of acceleration, a theta angle direction of acceleration, a phi angle direction of acceleration, and an amplitude of acceleration. The pattern detection component 420 may then compare the determined acceleration information for each of the plurality of acceleration peaks 310 to predetermined acceleration information that is known to be indicative of smoking behavior and then assign a probability of smoking behavior to each of the plurality of temporal segments 305 based at least in part on the comparison.

In accordance with various embodiments, the non-accelerometry data manager 425 may include circuitry, logic, hardware and/or software for processing data other than accelerometry data (e.g., data from a smoke detector or infrared heat sensor) for detecting smoking behavior of a user 105. The non-accelerometry data may be received by receiver 405-a via wired or wireless means from a sensor unit 110 having one or more sensors configured to detect smoke, heat, light, or sound such as a smoke detector, a heat sensor (e.g., infrared heat sensor), an optical sensor, or an audio sensor. Additionally, the non-accelerometry data manager 425 may process data that is manually input by the user 105 (e.g., medication dosage or consumption information). The non-accelerometry data may be used in conjunction with the accelerometry data to improve the specificity of the smoking behavior detection methods described herein.

For example, the non-accelerometry data manager 425 may detect the presence of heat or smoke from one or more sensor units 110 coupled with a user 105. The detection of heat or smoke alone may generally not be indicative of smoking behavior, but the non-accelerometry data manager 425 may correlate the detection of heat or smoke (or any other non-accelerometry data) with one or more patterns of movement detected by the pattern detection component 420 to more accurately detect smoking behavior. For example, it is more likely that a particular pattern of movement is indicative of smoking behavior if the non-accelerometry data manager 425 detects heat or smoke at the same time as the pattern detection component 420 detects a motion of both hands of the user 105 indicative of both hands coming together near the user's face (i.e., when the user 105 is lighting the cigarette). In a similar way, the non-accelerometry data manager 425 may detect an inhalation by the user 105 or the flicking sounds of a lighter and may correlate this data with patterns of movement detected by the pattern detection component 420.

The smoking behavior notification coordinator 430 may include circuitry, logic, hardware and/or software for generating a notification of smoking behavior once smoking behavior has been detected by the pattern detection component 420 or the non-accelerometry data manager 425. A notification of smoking behavior may be sent (via transmitter 415-a) to the user 105 or a responsible clinician for remote monitoring of the user 105. In addition to indicating that smoking behavior is detected, the smoking behavior notification coordinator 430 may generate additional information related to the smoking event including a confidence value or trend information (e.g., the number of smoking events detected throughout the day).

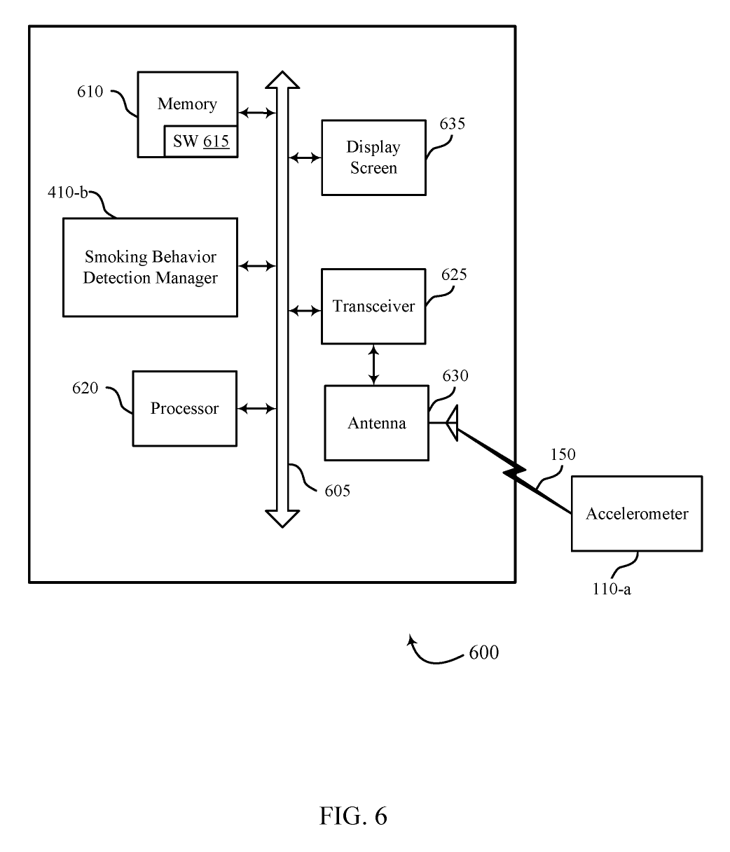

FIG. 6 shows a diagram of a device 600 that supports smoking behavior detection in accordance with various aspects of the present disclosure. The device 600 may be an example of aspects of device 400 as described with reference to FIG. 4, device 500 as described with reference to FIG. 5, or aspects of a sensor unit 110, a local computing device 115, a server 135, or a remote computing device 145 as described with reference to FIG. 1. The device 600 may communicate via wired or wireless means (e.g., wireless links 150) with a sensor unit 110-a (e.g., an accelerometer) that is physically coupled with a user 105, as described with reference to FIG. 1.

The device 600 may include a smoking behavior detection manager 410-b, which may be an example of aspects of the smoking behavior detection manager 410, 410-a described with reference to FIGS. 4 and 5. The device 600 may also include memory 610, a processor 620, a transceiver 625, and one or more antennas 630. Each of these components may communicate, directly or indirectly, with one another (e.g., via one or more buses 605). The memory 610 may be in electronic communication with the processor 620 and may include random access memory (RAM) and read only memory (ROM). The memory 610 may store computer-readable, computer-executable software (e.g., software 615) including instructions that, when executed, cause the processor 620 to perform various functions described herein (e.g., detecting one or more patterns of motion indicative of smoking behavior). In some cases, the software 615 may not be directly executable by the processor but may cause a computer (e.g., when compiled and executed) to perform functions described herein.

The processor 620 may include an intelligent hardware device, (e.g., a central processing unit (CPU), a microcontroller, or an application specific integrated circuit (ASIC). The transceiver 625 may communicate bi-directionally, via one or more antennas 630 or wired or wireless links 150 with one or more networks, as described with reference to FIG. 1. The transceiver 625 may also include a modem to modulate the packets and provide the modulated packets to the antennas 630 for transmission, and to demodulate packets received from the antennas 630.

The device 600 may also include a display screen 635 operable to display a notification conveying that smoking behavior has been detected. The display screen 635 may be an LCD screen or an LED screen for example.

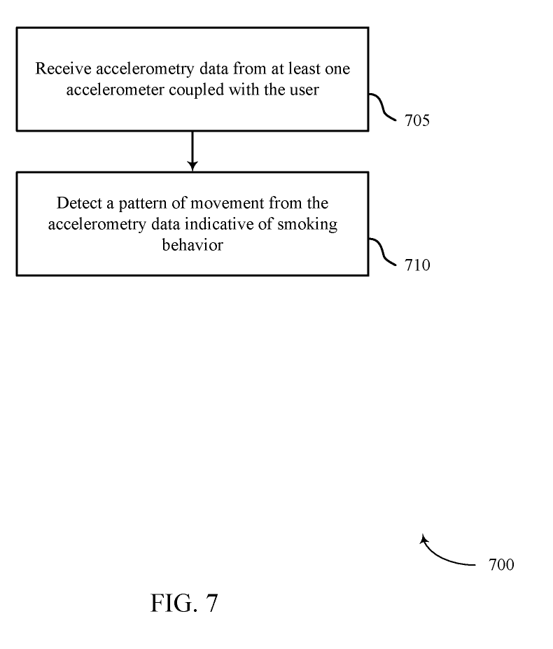

FIG. 7 shows a flowchart illustrating a method 700 for detecting smoking behavior of a user in accordance with various aspects of the present disclosure. The operations of method 700 may be implemented by aspects of one or more of device 400 as described with reference to FIG. 4, device 500 as described with reference to FIG. 5, device 600 as described with reference to FIG. 6, or a sensor unit 110, a local computing device 115, a server 135, or a remote computing device 145 as described with reference to FIG. 1. For example, the method 700 may be implemented by a smoking behavior detection manager 410. In some examples, the smoking behavior detection manager 410 may execute one or more sets of codes to control the functional elements of a device (e.g., processor 620 of device 600) to perform the functions described below. Additionally or alternatively, a device may perform aspects of the functions described below using special-purpose hardware.

At block 705, the method 700 may include receiving accelerometry data from at least one accelerometer coupled with the user 105. For example, the accelerometry data may be received by one or more sensor units 110 as described with reference to FIG. 1. With reference to FIGS. 4-6, the accelerometry data may be received via a receiver 405, 405-a, or one or more antennas 630 and a transceiver 625.

At block 710, the method 700 may also include detecting a pattern of movement from the accelerometry data indicative of smoking behavior. In accordance with various embodiments, any of devices 400, 500, or 600, or local computing device 115, server 135, remote computing device 145, sensor unit 110, and/or any of their modules or components may detect a pattern of movement indicative of smoking behavior. For example, with reference to FIGS. 4-6, the smoking behavior detection manager 410 may be operable to detect one or more patterns of movement indicative of smoking behavior. The pattern detection may include detecting one or more sub-patterns of movement that, either alone or in combination, are indicative of smoking behavior, as described with reference to FIG. 2. In some embodiments, detecting one or more sub-patterns of movement includes detecting an inhalation by the user 105 after detecting a motion of both hands of the user 105 indicative of both hands coming together near the user's face. Additionally or alternatively, the pattern detection may include processing the accelerometry data as described with reference to FIG. 3.

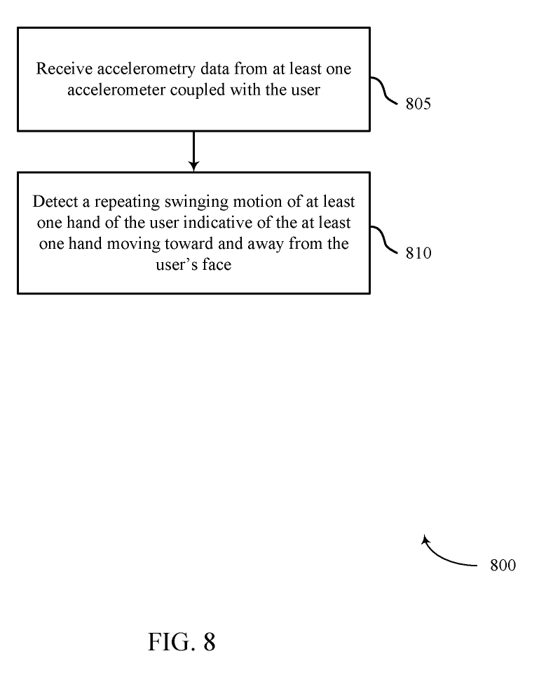

FIG. 8 shows a flowchart illustrating a method 800 for detecting smoking behavior of a user in accordance with various aspects of the present disclosure. The operations of method 800 may be implemented by aspects of one or more of device 400 as described with reference to FIG. 4, device 500 as described with reference to FIG. 5, device 600 as described with reference to FIG. 6, or a sensor unit 110, a local computing device 115, a server 135, or a remote computing device 145 as described with reference to FIG. 1. For example, the method 800 may be implemented by a smoking behavior detection manager 410. In some examples, the smoking behavior detection manager 410 may execute one or more sets of codes to control the functional elements of a device (e.g., processor 620 of device 600) to perform the functions described below. Additionally or alternatively, a device may perform aspects of the functions described below using special-purpose hardware.

At block 805, the method 800 may include receiving accelerometry data from at least one accelerometer coupled with the user 105. For example, the accelerometry data may be received by one or more sensor units 110 as described with reference to FIG. 1. With reference to FIGS. 4-6, the accelerometry data may be received via a receiver 405, 405-a, or one or more antennas 630 and a transceiver 625.

At block 810 the method 800 may also include detecting a repeating swinging motion of at least one hand of the user 105 indicative of the at least one hand moving toward and away from the user's face, which may be an example of motion 215 described with reference to FIG. 2. In accordance with various embodiments, any of devices 400, 500, or 600, or local computing device 115, server 135, remote computing device 145, sensor unit 110, and/or any of their modules or components may detect a repeating swinging motion of at least one hand of the user 105. For example, with reference to FIGS. 4-6, the smoking behavior detection manager 410 may be operable to detect a repeating swinging motion of at least one hand of the user 105.

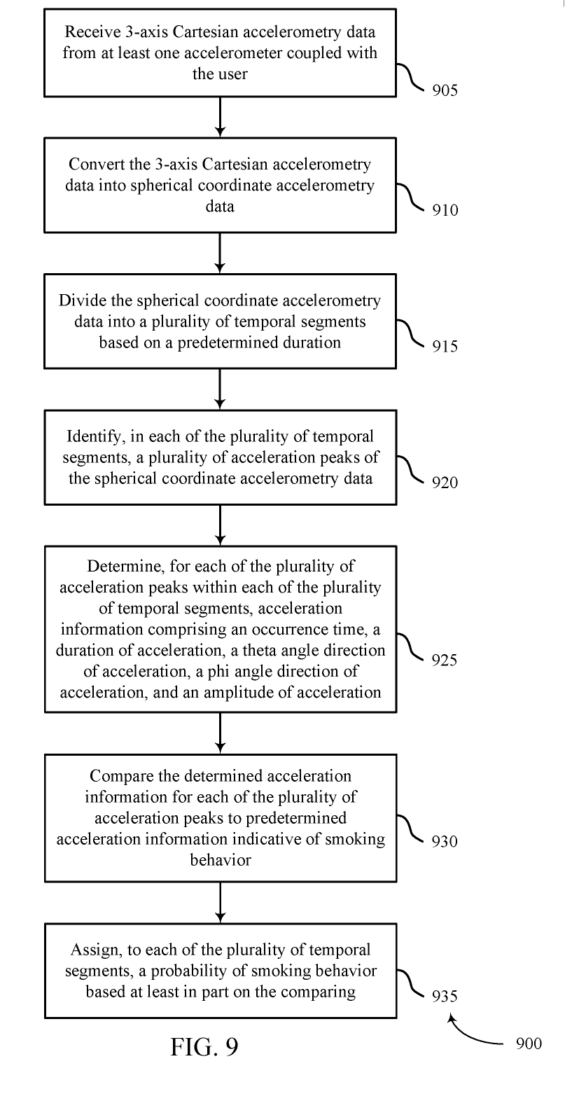

FIG. 9 shows a flowchart illustrating a method 900 for detecting smoking behavior of a user in accordance with various aspects of the present disclosure. The operations of method 900 may be implemented by aspects of one or more of device 400 as described with reference to FIG. 4, device 500 as described with reference to FIG. 5, device 600 as described with reference to FIG. 6, or a sensor unit 110, a local computing device 115, a server 135, or a remote computing device 145 as described with reference to FIG. 1. For example, the method 900 may be implemented by a smoking behavior detection manager 410. In some examples, the smoking behavior detection manager 410 may execute one or more sets of codes to control the functional elements of a device (e.g., processor 620 of device 600) to perform the functions described below. Additionally or alternatively, a device may perform aspects of the functions described below using special-purpose hardware.

At block 905, the method 900 may include receiving 3-axis Cartesian accelerometry data from at least one accelerometer coupled with the user 105. The accelerometry data may be received by one or more sensor units 110 as described with reference to FIG. 1. With reference to FIGS. 4-6, the accelerometry data may be received via a receiver 405, 405-a, or one or more antennas 630 and a transceiver 625.

At block 910, the method 900 may further include converting the 3-axis Cartesian accelerometry data into spherical coordinate accelerometry data. In accordance with various embodiments, any of devices 400, 500, or 600, or local computing device 115, server 135, remote computing device 145, sensor unit 110, and/or any of their modules or components may convert the accelerometry data into spherical coordinate data. For example, with reference to FIGS. 4-6, the smoking behavior detection manager 410 may be operable to perform the conversion.

At block 915, the method 900 may further include dividing the spherical coordinate accelerometry data into a plurality of temporal segments based on a predetermined duration. For example, with reference to FIG. 3, the accelerometry data may be divided into a plurality of temporal segments 305.

At block 920, the method 900 may further include identifying, in each of the plurality of temporal segments, a plurality of acceleration peaks of the spherical coordinate accelerometry data. Referring again to FIG. 3, one or more acceleration peaks 310 may be identified by any of devices 400, 500, or 600, or local computing device 115, server 135, remote computing device 145, sensor unit 110, and/or any of their modules or components.

At block 925, the method 900 may further include determining, for each of the plurality of acceleration peaks within each of the plurality of temporal segments, acceleration information comprising an occurrence time, a duration of acceleration, a theta angle direction of acceleration, a phi angle direction of acceleration, and an amplitude of acceleration. In accordance with various embodiments, any of devices 400, 500, or 600, or local computing device 115, server 135, remote computing device 145, sensor unit 110, and/or any of their modules or components may be operable to determine the acceleration information as described.

At block 930, the method 900 may further include comparing the determined acceleration information for each of the plurality of acceleration peaks to predetermined acceleration information indicative of smoking behavior. The comparing may be performed by any of devices 400, 500, or 600, or local computing device 115, server 135, remote computing device 145, sensor unit 110, and/or any of their modules or components.

At block 935, the method 900 may further include assigning, to each of the plurality of temporal segments, a probability of smoking behavior based at least in part on the comparing. As described with reference to FIG. 3, the probability assignment may be based on a machine learning algorithm and may be performed by any of devices 400, 500, or 600, or local computing device 115, server 135, remote computing device 145, sensor unit 110, and/or any of their modules or components.

It should be noted that these methods describe possible implementation, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein. Thus, aspects of the disclosure may provide for smoking behavior detection.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical (PHY) locations. Also, as used herein, including in the claims, “or” as used in a list of items (for example, a list of items prefaced by a phrase such as “at least one of” or “one or more”) indicates an inclusive list such that, for example, a list of at least one of A, B, or C means A or B or C or AB or AC or BC or ABC (i.e., A and B and C).

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

What is claimed is:

1. A method for detecting smoking behavior of a user, comprising:
receiving accelerometry data from at least one accelerometer coupled with the user; and
detecting a pattern of movement from the accelerometry data indicative of smoking behavior.

2. The method of claim 1, wherein detecting the pattern of movement comprises detecting a repeating swinging motion of at least one hand of the user indicative of the at least one hand moving toward and away from the user's face.

3. The method of claim 1, wherein detecting the pattern of movement comprises detecting a plurality of sub-patterns of movement in a sequence indicative of smoking behavior.

4. The method of claim 3, wherein detecting the plurality of sub-patterns of movement comprises detecting a vibration motion of at least one hand of the user indicative of the user flicking at least a portion of the at least one hand after detecting a repeating swinging motion of the at least one hand indicative of the at least one hand moving toward and away from the user's face.

5. The method of claim 3, wherein detecting the plurality of sub-patterns of movement comprises detecting a repeating swinging motion of at least one hand of the user indicative of the at least one hand moving toward and away from the user's face after detecting a motion of both hands of the user indicative of both hands coming together away from the user's face.

6. The method of claim 3, wherein detecting the plurality of sub-patterns of movement comprises detecting a repeating swinging motion of at least one hand of the user indicative of the at least one hand moving toward and away from the user's face after detecting a motion of both hands of the user indicative of both hands coming together near the user's face.

7. The method of claim 3, wherein detecting the plurality of sub-patterns of movement comprises detecting an inhalation by the user after detecting a motion of both hands of the user indicative of both hands coming together near the user's face.

8. The method of claim 1, wherein the accelerometry data comprises 3-axis Cartesian accelerometry data; and wherein detecting the pattern of movement from the 3-axis Cartesian accelerometry data comprises:
converting the 3-axis Cartesian accelerometry data into spherical coordinate accelerometry data;
dividing the spherical coordinate accelerometry data into a plurality of temporal segments based on a predetermined duration;
identifying, in each of the plurality of temporal segments, a plurality of acceleration peaks of the spherical coordinate accelerometry data;
determining, for each of the plurality of acceleration peaks within each of the plurality of temporal segments, acceleration information comprising an occurrence time, a duration of acceleration, a theta angle direction of acceleration, a phi angle direction of acceleration, and an amplitude of acceleration;
comparing the determined acceleration information for each of the plurality of acceleration peaks to predetermined acceleration information indicative of smoking behavior;
assigning, to each of the plurality of temporal segments, a probability of smoking behavior based at least in part on the comparing.

9. The method of claim 1, wherein the at least one accelerometer is coupled with a wrist of the user.

10. The method of claim 1, further comprising detecting thermal energy with an infrared sensor.

11. The method of claim 1, further comprising detecting smoke with a smoke detector.

12. An apparatus for detecting smoking behavior of a user, comprising:
a processor;
memory in electronic communication with the processor; and
instructions stored in the memory and operable, when executed by the processor, to cause the apparatus to:
receive accelerometry data from at least one accelerometer; and
detect a pattern of movement from the accelerometry data indicative of smoking behavior.

13. The apparatus of claim 12, wherein the instructions are operable to cause the processor to detect a repeating swinging motion of at least one hand of the user indicative of the at least one hand moving toward and away from the user's face.

14. The apparatus of claim 12, wherein the instructions are operable to cause the processor to detect a plurality of sub-patterns of movement in a sequence indicative of smoking behavior.

15. The apparatus of claim 14, wherein the instructions are operable to cause the processor to detect a vibration motion of at least one hand of the user indicative of the user flicking at least a portion of the least one hand after detecting a repeating swinging motion of the at least one hand indicative of the at least one hand moving toward and away from the user's face.

16. The apparatus of claim 14, wherein the instructions are operable to cause the processor to detect a repeating swinging motion of at least one hand of the user indicative of the at least one hand moving toward and away from the user's face after detecting a motion of both hands of the user indicative of both hands coming together away from the user's face.

17. The apparatus of claim 14, wherein the instructions are operable to cause the processor to detect a repeating swinging motion of at least one hand of the user indicative of the at least one hand moving toward and away from the user's face after detecting a motion of both hands of the user indicative of both hands coming together near the user's face.

18. The apparatus of claim 14, wherein the instructions are operable to cause the processor to detect an inhalation by the user after detecting a motion of both hands of the user indicative of both hands coming together near the user's face.

19. The apparatus of claim 12, wherein the instructions are operable to cause the processor to transmit a notification conveying that smoking behavior has been detected after a pattern of movement indicative of smoking behavior has been detected.

20. A system for detecting smoking behavior of a user, comprising:
at least one wearable apparatus coupled with the user, the at least one wearable apparatus comprising at least one accelerometer;
a computing apparatus in electronic communication with the at least one wearable apparatus, the computing apparatus configured to detect a pattern of movement from accelerometry data from the at least one accelerometer indicative of smoking behavior; and
a display in electronic communication with the computing apparatus, the display configured to display a notification conveying that smoking behavior has been detected after a pattern of movement indicative of smoking behavior has been detected.
